(19) United States
(12) Patent Application Publication
(10) Pub. No.: US 2017/0027849 A1
(43) Pub. Date: Feb. 2, 2017

(54) Oral Care Compositions Containing Amps Polymers, Copolymers, Or Cross Polymers

(71) Applicant: The Procter & Gamble Company, Cincinnati, OH (US)

(72) Inventors: Sanjeev Midha, Mason, OH (US); Steven Hamilton Hoke, II, West Chester, OH (US); Laura H. Lucas, Cincinnati, OH (US); Lawrence Edward Dolan, Cincinnati, OH (US); Michael Jude LeBlanc, Cincinnati, OH (US); Karen Lehnhoff, Cincinnati, OH (US); Eva Schneiderman, Mason, OH (US)

(21) Appl. No.: 15/210,916

(22) Filed: Jul. 15, 2016

Related U.S. Application Data
(60) Provisional application No. 62/197,721, filed on Jul. 28, 2015.

(51) Int. Cl. A61K 8/88; A61K 8/21; A61K 8/31; A61K 8/81; A61K 8/34; A61Q 11/00; A61K 8/22

(52) U.S. Cl. CPC A61K 8/88; A61Q 11/00; A61K 8/21; A61K 8/22; A61K 8/8141; A61K 8/342; A61K 8/31; A61K 2800/28

(57) ABSTRACT

Oral care compositions containing a peroxide and a 2-acrylamido-2-methylpropane sulfonic acid polymers, copolymers, or cross polymers. The oral care compositions can also contain a fluoride ion source and a flavor.

### FIELD OF THE INVENTION

The present invention relates to an oral care composition. More particularly an oral care composition containing fluoride salts, a peroxide source, a flavor and a 2-acrylamido-2-methylpropane sulfonic acid (AMPS) polymers, copolymers, or cross polymers.

### BACKGROUND OF THE INVENTION

Oral care compositions, including dentifrice compositions, can contain fluoride salts, peroxide, abrasives, and flavors, to clean teeth, freshen breath, and maintain the aesthetics and health of the oral cavity, including the teeth and gums.

It can be especially desirable to have toothpastes with relatively high levels of peroxide, for instance greater than about 1%, in order to achieve enhanced whitening. It is difficult to formulate oral care compositions with high levels of peroxide. First, peroxide can be unstable.

When peroxide breaks down, it forms oxygen, water, and radicals. The excess gas can cause swelling and bursting of primary packaging and the radicals can cause the entire oral care composition, including the actives and flavors, to break down to a composition with a waterlike viscosity and decreased efficacy.

Peroxide can also have an off taste and it is important that compositions with peroxide, especially relatively high levels of peroxide, have a strong flavor display. Furthermore, flavors can degrade, especially if the peroxide is unstable. One solution is to use high levels of flavor. However, using high levels of flavors, especially in combination with peroxide, can irritate a user's mouth and can be expensive.

As such, there is a need for an improved oral care composition that has a relatively high level of stable peroxide, sufficient rheology, and a strong flavor display.

### SUMMARY OF THE INVENTION

An oral care composition comprising: (a) a fluoride ion source selected from the group consisting of stannous fluoride, sodium fluoride, potassium fluoride, amine fluoride, sodium monofluorophosphate, indium fluoride, amine fluorides, and combinations thereof; (b) greater than about 0.5% peroxide selected from the group consisting of hydrogen peroxide, urea peroxide, calcium peroxide, sodium peroxide, zinc peroxide, or combinations thereof; and (c) a 2-acrylamido-2-methylpropane sulfonic acid polymer, copolymer, and/or cross polymer.

An oral care composition comprising: (a) greater than about 0.5% peroxide selected from the group consisting of hydrogen peroxide, urea peroxide, calcium peroxide, sodium peroxide, zinc peroxide, or combinations thereof; (b) a 2-acrylamido-2-methylpropane sulfonic acid polymer, copolymer, and/or cross polymer; (c) a flavor; wherein the oral care composition has an average total flavor peak area of greater than about 10% greater than the average total flavor peak area from the gel network chassis according to the Total Flavor Display Test by Headspace GC-MS.

An oral care composition comprising: (a) a fluoride ion source selected from the group consisting of stannous fluoride, sodium fluoride, potassium fluoride, amine fluoride, sodium monofluorophosphate, indium fluoride, amine fluorides, and combinations thereof; (b) greater than about 0.5% peroxide selected from the group consisting of hydrogen peroxide, urea peroxide, calcium peroxide, sodium peroxide, zinc peroxide, or combinations thereof; (c) an abrasive selected from the group consisting of silica, polyorganosilsequioxane, calcium pyrophosphate, poly(methyl methacrylate), calcium carbonate, dicalcium phosphate, barium sulfate, and combinations thereof; (d) greater than about 10% water; (e) a flavor; (f) a sweetener; wherein the oral care composition has an average total flavor peak area of greater than about 10% greater than the average total flavor peak area from the gel network chassis containing the exact same flavor at the same level as determined by the Total Flavor Display Test by Headspace GC-MS; wherein at least about 70% of peroxide remains after 4 weeks according to the Peroxide stability test; and wherein the oral care composition is a dentifrice.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1A shows the molecular structure for AMPS;

FIG. 1B shows the molecular structure for polyacrylate crosspolymer-6; and

FIG. 2 compares the total flavor display in three examples;

FIG. 3A is a photo at 600X magnification of a composition containing Tospearl® Silicone Microspheres suspended in a formulation thickened with polyacrylate crosspolymer-6;

FIG. 3B is a photo at 600× magnification of a composition containing Tospearl® Silicone Microspheres suspended in a formulation containing polyacrylate crosspolymer-6 and a fatty alcohol; and

FIG. 3C is a photo at 600× magnification of a composition containing Tospearl® Silicone Microspheres suspended in a formulation containing a fatty alcohol and a surfactant system.

### DETAILED DESCRIPTION OF THE INVENTION

Oral care compositions can include fluoride, peroxide, abrasives, flavors, and other ingredients to provide benefits like reducing plaque and tartar, preventing cavities, preventing and reversing gingivitis, building protection against sensitivity, freshening bad breath, and whitening teeth. In order to provide these benefits, oral care compositions can contain fluoride salts, peroxide, abrasives, and flavors.

Some consumers are particularly interested in a product that contains a relatively high amount of peroxide, for instance greater than about 1%, in order to enhance removal of intrinsic and extrinsic stains.

However, oral care compositions, especially those containing a peroxide source and/or a fluoride ion source, can be unstable and difficult to formulate. For example, peroxides readily decompose, such as hydrogen peroxide which decomposes to form water and oxygen. When peroxide decomposes it can be less efficacious, produce excess gas, which can cause swelling and bursting of primary packaging, and can destroy the viscosity and efficacy of the other components in the oral care composition, including flavors. If the flavor degrades, it is more difficult to mask the taste of peroxide, as well as other off-tasting ingredients.

Because peroxide and its degradants can have an off taste, it can be important for compositions with peroxide, especially relatively high levels of peroxide, to have a strong flavor display. However, flavors can degrade, especially in the presence of unstable peroxides. One way to obtain a strong flavor display is use high levels of flavor. However, using high levels of flavors can irritate a user's mouth, especially in the presence of peroxide, which can also be irritating, and flavors are expensive. The oral mucosal can be especially sensitive due to its moist linings (lamina propria) and large number of nerve endings, including special sensory endings on the tongue specially adapted for taste.

Therefore, it can be desirable to add a component to the oral care composition that can help stabilize peroxide, maintain the original viscosity and promote the flavor display, which can allow less flavor to be used in the composition. In one example, the oral care composition can contain an AMPS polymer, co-polymer, and/or crosspolymer. In one example, the oral care composition can contain polyacrylate crosspolymer-6 (commercially available as SepiMAX™ ZEN from SEPPIC S.A., a subsidiary of the Air Liquide group, Puteaux Cedex, France). The viscosity of the oral care composition can be from about 10 to about 50 BKUs. In another example, the viscosity of the oral care composition can be from about 20 to about 30 BKUs.

In another example, the oral care composition can be a dentifrice and can contain polyacrylate crosspolymer-6, fluoride salts, a flavor, a peroxide source, and an abrasive. In one example, the composition can contain at least about 1.6% polyacrylate crosspolymer-6 and in another example at least about 3.2% polyacrylate crosspolymer-6. In another example the oral care composition can contain greater than about 1% peroxide. In another example, the oral care composition can be a dentifrice and can contain a fatty alcohol. In one example the oral care composition can contain stannous fluoride, in another example the oral care composition can contain sodium fluoride, and in another example the oral care composition can contain monofluorophosphate.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. (i.e. room temperature) unless otherwise specified.

The composition can contain, consist of, or consist essentially of, the essential elements and limitations of the invention described herein, as well as any additional or optional ingredients, components, or limitations described herein or otherwise useful in oral care compositions.

As used herein, the word “include,” and its variants, are intended to be non-limiting, such that recitation of items in a list is not to the exclusion of other like items that may also be useful in the materials, compositions, devices, and methods of this invention.

As used herein, the articles “a” and “an” are understood to mean one or more of the material that is claimed or described, for example, “an abrasive” or “a surfactant”.

As used herein, the word ^{or} when used as a connector of two or more elements is meant to include the elements individually and in combination; for example X or Y, means X or Y or both.

By “oral care composition”, as used herein, is meant a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice”, as used herein, includes tooth or subgingival paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

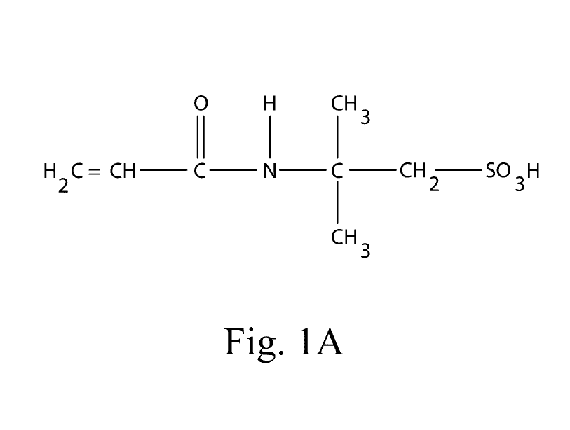

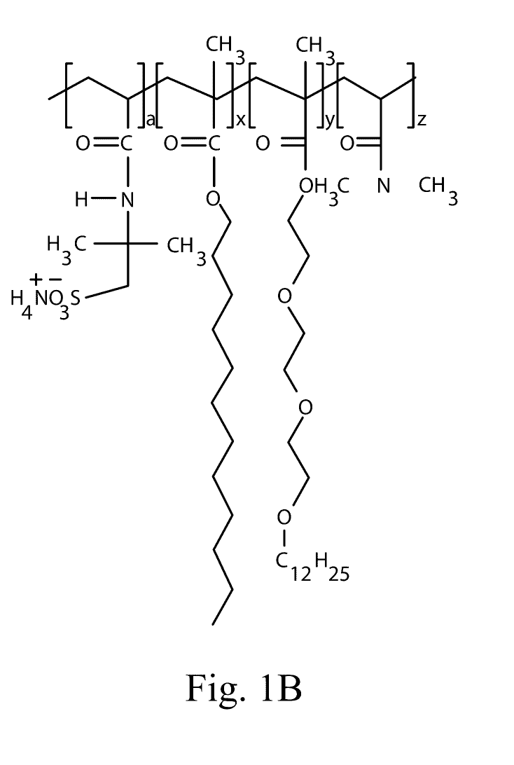

The term “water”, as used herein, refers to deionized water, unless otherwise specified. Oral care compositions containing AMPS polymers, copolymers and crosspolymers can have improved flavor display and viscosity. FIG. 1A shows the molecular structure for AMPS. In one example, the composition can contain polyacrylate crosspolymer-6. The molecular structure of polyacrylate crosspolymer-6 is shown in FIG. 1B. Non-limiting examples of polymers, copolymers and crosspolymers synthesized from AMPS can include hydroxyethyl acrylate/sodium acryloyldimethyl taurate copolymer (commercially available as Sepinov™ EMT-10 from SEPPIC S.A.), ammonium acryloyldimethyl taurate/vinyl pyrrolidone copolymer (commercially available as Aristoflex® AVC from Clariant International LTD, Muttenz, Switzerland), ammonium acryloyldimethyltaurate/beheneth-25 methacrylate crosspolymer (commercially available as Aristoflex® HMB, Clariant International LTD), sodium acrylate/sodium acryloyldimethyltaurate copolymer (a component of Sepigel EG and Simulgel SMS 88, SEPPIC S.A.), acrylamide/sodium acryloyldimethyltaurate copolymer (a component of Simulgel 600 and Simulgel 600 PHA, SEPPIC S.A.), polyacrylate crosspolymer-6 (commercially available as SepiMAX™ ZEN from SEPPIC S.A.), and combinations thereof.

In some examples, it can be difficult to find a polymer, particularly an AMPS polymer, copolymer, or crosspolymer that is compatible with common oral care composition components such as fluoride salts, metal salts, peroxides, and/or abrasives. If the polymer is incompatible with oral care components, it may not help thicken the composition.

In one example, the oral care compositions can contain a polyacrylate crosspolymer-6. The molecular structure of polyacrylate crosspolymer-6 is shown in FIG. 1B. Polyacrylate crosspolymer-6 is a copolymer of Ammonium 2-methyl-2-[(1-oxo-2-propenyl)amino]-1-propanesulfonate, N,N, Dimethyl-2-acrylamide, Poly(oxy-1,2-ethanediyl), alpha-(2-methyl-1-oxo-2-propenyl0-omega-(dodecyloxy) and Methyl-2-propenoic acid dodecyl ester monomers.

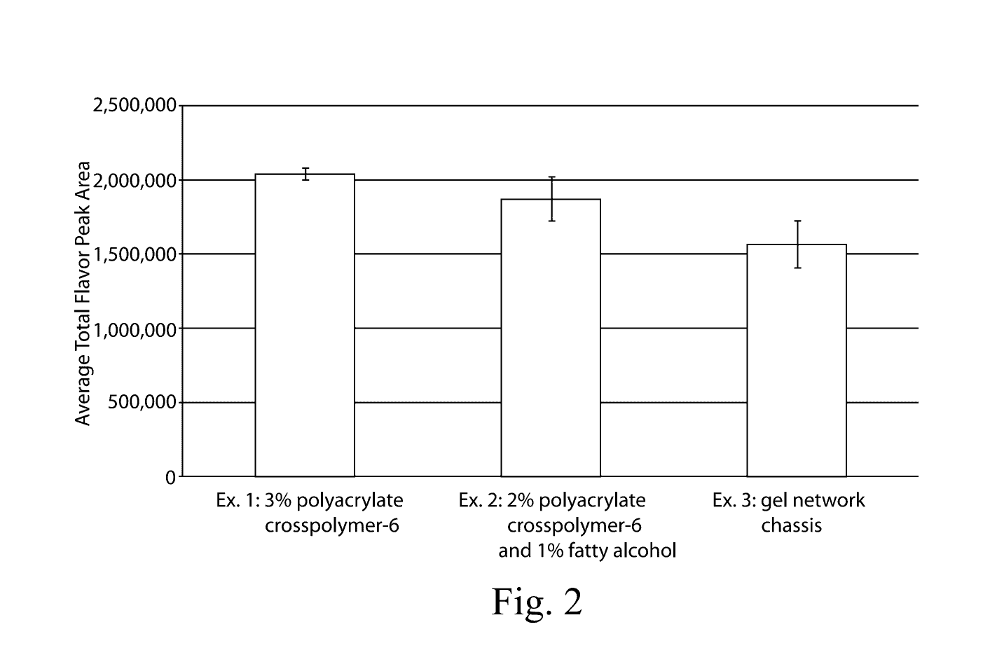

FIG. 2 compares the average total flavor display in Examples 1, 2, and 3, described hereafter. The error bars in FIG. 2 indicate the standard error of the mean. A summary of the

Examples is below:
Example 1: 3% polyacrylate crosspolymer-6;
Example 2: 2% polyacrylate crosspolymer-6 and 1% fatty alcohol; and
Example 3 (gel network): a fatty alcohol chassis without polyacrylate crosspolymer-6.

The table below shows the peak areas and percentages of flavor peak area increase relative to the gel network example.

| | Average Total | % Flavor Display Increase |
|---|---|---|
| | Flavor Peak Area | vs. Gel Network |
| Example 1 | 2,039,988 | 30.3 |
| Example 2 | 1,873,362 | 19.7 |
| Example 3 | 1,565,618 | |

Example 1, with 3% polyacrylate crosspolymer-6, had the strongest flavor display, as evidenced by the largest headspace measurement. For Example 1, the average total flavor peak area was over 30% more intense than the gel network determined by the Total Flavor Display Test by Headspace GC-MS. Example 1 gives very good flavor display, while using 3% flavor. It was surprising that crosspolymer-6 both helped stabilize the peroxide and improve the flavor display because traditional compositions that stabilize peroxide, such as the fatty alcohol based thickening network (gel network) of Example 3, tend to suppress the total flavor display. In comparison to Example 1, the flavor display of Example 3 was relatively low, as it was about 1.5 to 1.6 million (about 1,566,000) area units. The headspace flavor display can be determined by using the Total Flavor Display Test by Headspace GC-MS as described hereafter.

The flavor display for Example 2 is also acceptable and it can sometimes be desirable to use both a fatty alcohol and polyacrylate crosspolymer-6. The flavor display for Example 2 was about 20% more intense than the flavor display from the gel network. In some examples, it can be preferable to include both polyacrylate crosspolymer-6 and a fatty alcohol. First, polyacrylate crosspolymer-6 can be expensive. Furthermore, oral care compositions that do not contain a fatty alcohol can have a rheology that is less desirable. For instance, if the composition is a dentifrice, the rheology can be stringy and doughy and the nurdle can be lumpy when applied to a toothbrush. In some examples, using both polyacrylate crosspolymer-6 and a fatty alcohol can improve the overall rheology, deliver very good flavor display as well as lower cost.

Example 3 can be advantageous because fatty alcohol gel networks can help stabilize peroxide. However, the total flavor display is not as good as Examples 1 and 2.

Polyacrylate crosspolymer-6 can also improve the viscosity of compositions, in particular, compositions with relatively high levels of peroxide. Examples 4-8 were made as described hereafter and the viscosity was measured using the Brookfield Viscosity Test as described hereafter. A summary of Examples 4-8 and the viscosity data are in the table below.

| | Ex. 4 | Ex. 5 | Ex 6 | Ex 7 | Ex 8 |
|---|---|---|---|---|---|
| SepiMAX ™ ZEN (wt. %) | 3.2 | 1.6 | 0 | 1.6 | 0 |
| Lanette ® W (wt. %) | 0 | 1.6 | 3.2 | 0 | 1.6 |
| Brookfield Viscosity (BKUs) | 26 | 23 | 4 | 5 | 3 |

Example 4 had the highest viscosity and in some examples this is desirable. However, Example 4 has a rheology that is not preferred by some consumers because it can be stringy and the nurdle on the toothbrush can be lumpy and doughy. Example 5, with SepiMAX™ ZEN and Lanette® W, had a viscosity that was both acceptable and very close to the viscosity of Example 4, which contains twice the amount of SepiMAX™ ZEN. However, examples 7 and 8, which have 1.6% SepiMAX™ ZEN or 1.6% Lanette® W, respectively, have viscosities that can be considered much too low to make acceptable toothpastes. It is surprising that combining these two compositions, as was done in Example 5, would more than quadruple the viscosity.

Lanette® W is a mixture of fatty alcohols and surfactant and is a commercially available mixture of approximately 45% cetyl alcohol, 45% stearyl alcohol, and 10% sodium lauryl sulfate (available from BASF, Florham Park, N.J.). Formulations have been made that have an acceptable viscosity that have about 14% Lanette® W. However, to incorporate Lanette® W into a dentifrice composition, it must be heated, which increases the manufacturing time and cost. Therefore, using a lower level of Lanette® W can be desirable, like Example 5, which contains 1.6% Lanette® W. Additionally, adding polyacrylate crosspolymer-6 can benefit the flavor display, as discussed above.

In one example the oral care composition can have a viscosity from about 5 BKUs to about 70 BKUs, in another example from about 10 BKUs to about 45 BKUs, in another example from about 12 BKUs to about 40 BKUs, in another example from about 15 BKUs to about 35 BKUs, in another example from about 18 BKUs to about 30 BKUs, in another example from about 20 BKUs to about 28 BKUs, and in another example from about 22 BKUs to about 25 BKUs. In another example the oral care composition can have a viscosity form about 7 BKUs to about 150 BKUs, in another example from about 10 BKUs to about 100 BKUs, in another example from about 12 BKUs to about 90 BKUs, in another example from about 15 BKUs to about 80 BKUs, and in another example from about 20 BKUs to about 75 BKUs. Viscosity can be measured by the Brookfield Viscosity Test as described hereafter.

Polyacrylate crosspolymer-6 can also improve the stability of peroxide in oral care compositions, in particular, compositions with relatively high levels of peroxide. Examples 4-6 were made as described hereafter and the % peroxide was measured using the Peroxide Stability Test as described hereafter. A summary of Examples 4-6 and the peroxide stability data are in the table below.

| | Ex. 4 | Ex. 5 | Ex 6 |
|---|---|---|---|
| SepiMAX ™ ZEN (wt. %) | 3.2 | 1.6 | 0 |
| Lanette ® W (wt. %) | 0 | 1.6 | 3.2 |
| % Peroxide remaining after 4 | 99.7 | 98.7 | 95.3 |
| weeks at 40° C. | | | |
| % Peroxide remaining after 8 | 96 | 96 | 95.3 |
| weeks at 40° C. | | | |

Examples 4, 5, and 6 all had good peroxide stability and the peroxide could be stable enough for the product's shelf life. It was found that compositions containing polyacrylate crosspolymer-6, such as Examples 4 and 5, also have stable peroxide.

In one example at least about 70% of peroxide remains after 4 weeks at 40° C. according to the Peroxide Stability Test, in another example at least about 75%, in another example at least about 80%, in another example at least about 85%, in another example at least about 90%, in another example at least about 94%, in another example at least about 97%, and in another example at least about 98%. In one example from about 55% to about 100% of peroxide remains after 4 weeks at 40° C. according to the Peroxide Stability Test, in another example from about 65% to about 99%, in another example from about 75% to about 98%, in another example from about 80% to about 97%, and in another example from about 90% to about 95%.

In one example at least about 68% of peroxide remains after 8 weeks at 40° C. according to the Peroxide Stability Test, in another example at least about 72%, in another example at least about 78%, in another example at least about 83%, in another example at least about 88%, in another example at least about 90%, in another example at least about 93%, and in another example at least about 95%. In one example from about 50% to about 100% of peroxide remains after 8 weeks at 40° C. according to the Peroxide Stability Test, in another example from about 60% to about 99%, in another example from about 70% to about 98%, in another example from about 80% to about 97%, and in another example from about 90% to about 95%.

In another example at least about 40% of peroxide remains after 13 weeks or 26 weeks at 40° C. according to the Peroxide Stability Test, in another example at least about 50%, in another example at least about 60%, in another example at least about 70%, in another example at least about 80%, and in another example at least about 90%. In another example from 45% to about 95% of peroxide remains after 13 weeks or 26 weeks at 40° C. according to the Peroxide Stability Test, in another example from about 55% to about 90%, in another example from about 60% to about 85%, and in another example from about 65% to about 80%.

In one example the oral care composition can have a shelf life, when stored below 40° C., of at least 6 months, in another example at least 1 year, in another example at least 18 months, in another example at least 2 years, in another example at least 30 months, and in another example at least 3 years. In another example, the shelf life can be from about 6 months to about 5 years, in another example from about 1 year to about 3 years, and in another example from about 1.5 years to about 2.5 years.

In one example, the oral care composition can have an average total flavor peak area of greater than about 10% greater than the average total flavor peak area from the gel network chassis as determined by the Total Flavor Display Test by Headspace GC-MS, in another example greater than about 12% greater, in another example greater than about 15% greater, in another example greater than about 17% greater, in another example greater than about 20% greater, in another example greater than about 25% greater, in another example greater than about 28% greater, and in another example more than 30% greater than the average total flavor peak area from the gel network chassis as determined by the Total Flavor Display Test by Headspace GC-MS.

The oral care composition can have a pH from about 2 to about 8, in another example from about 3 to about 7, in another example from about 4 to about 6, and in another example from about 4.5 to about 5.5. pH can be measured using the pH test method as described hereafter.

In one example, the oral care composition can contain from about 0.1% to about 10% AMPS polymer, copolymer, or crosspolymer, in another example from about 0.5% to about 7%, in another example from about 1% to about 5%, in another example from about 1.2% to about 4%, and in another example from about 1.6% to about 3.5%.

In one example, the oral care composition can contain from about 0.3% to about 5% polyacrylate crosspolymer-6, in another example from about 0.5% to about 4.5%, in another example from about 1% to about 4%, in another example from about 1.5% to about 3.75%, in another example from about 2% to about 3.5%, in another example from about 2.5% to about 3.25%, and in another example from about 2.75% to about 3.1%. In one example, the oral care composition can contain from about 1% to about 6% polyacrylate crosspolymer-6, in another example from about 2% to about 4%, in another example from about 2.5% to about 3.7%, and in another example from about 3% to about 3.5%. In another example, the oral care composition can contain from about 0.2% to about 3% polyacrylate crosspolymer-6, in another example from about 0.8% to about 2.5%, in another example from about 1% to about 2.2%, and in another example from about 1.2% to about 2%.

In addition to the AMPS polymer, copolymer, or crosspolymer compositions may contain fatty amphiphiles. Fatty amphiphiles can enhancement product viscosity and can provide the ability to adjust formulation texture for enhancement of consumer in-use experience.

As used herein, “fatty amphiphile” refers to a compound having a hydrophobic tail group and a hydrophilic head group which does not make the compound water soluble (immiscible), wherein the compound also has a net neutral charge at the pH of the oral composition. The fatty amphiphile can be selected from the group consisting of fatty alcohols, alkoxylated fatty alcohols, fatty phenols, alkoxylated fatty phenols, fatty amides, alkyoxylated fatty amides, fatty amines, fatty alkylamidoalkylamines, fatty alkyoxyalted amines, fatty carbamates, fatty amine oxides, fatty acids, alkoxylated fatty acids, fatty diesters, fatty sorbitan esters, fatty sugar esters, methyl glucoside esters, fatty glycol esters, mono, di- and tri-glycerides, polyglycerine fatty esters, alkyl glyceryl ethers, propylene glycol fatty acid esters, cholesterol, ceramides, fatty silicone waxes, fatty glucose amides, phospholipids, and combinations thereof. Suitable fatty amphiphiles include a combination of cetyl alcohol and stearyl alcohol.

The oral care compositions may comprise fatty amphiphile in an amount from about 0.05% to about 30%, preferably from about 0.1% to about 20%, and more preferably from about 0.5% to about 10%, by weight of the oral care composition.

In one example, the oral care composition can contain a fatty alcohol in an amount from about 0.1% to about 10%, in another example from about 0.5% to about 7%, in another example from about 1% to about 5%, and in another example from about 1.4% to about 3%. In one example the oral care composition can contain greater than 0% and less than about 5% fatty alcohol, in another example greater than 0% and less than 4% fatty alcohol, in another example greater than 0% and less than 3% fatty alcohol, and in another example greater than 0% and less than 2% fatty alcohol.

The oral care composition can contain one or more surfactants. The surfactant is typically water soluble or miscible in the solvent or oral carrier. Suitable surfactants include anionic, zwitterionic, amphoteric, cationic, and nonionic surfactants. In one embodiment, anionic surfactants such as sodium lauryl sulfate, are preferred. The surfactants may be a combination of more than one type of surfactants, such as an anionic and nonionic surfactant. Suitable solvents for the present invention can include water, edible polyhydric alcohols such as glycerin, diglycerin, triglycerin, sorbitol, xylitol, butylene glycol, erythritol, polyethylene glycol, propylene glycol, and combinations thereof.

Actives and other ingredients may be categorized or described herein by their cosmetic benefit, therapeutic benefit, or their postulated mode of action or function. However, it is to be understood that the active and other ingredients useful herein can, in some instances, provide more than one cosmetic benefit, therapeutic benefit, function, or can operate via more than one mode of action. Therefore, classifications herein are made for the sake of convenience and are not intended to limit an ingredient to the particularly stated function(s) or activities listed.

A metal salt includes zinc salts, stannous salts, potassium salts, copper salts, alkali metal bicarbonate slats, and combinations thereof. Metal salts have a wide range of functions from antimicrobial agents to sensitivity agents or buffers. The oral care compositions of the present invention may contain metal salt in an amount from about 0.05% to about 11%, from about 0.5% to about 7%, or from about 1% to about 5%, by total weight of the oral care composition. Some metal salts which may be used in the present invention, such as zinc chloride, zinc citrate, copper gluconate, and zinc gluconate, are also associated with an off taste described as dirty, dry, earthy, metallic, sour, bitter, and astringent.

It is common to have a fluoride compound present in dentifrices and other oral care compositions in an amount sufficient to give a fluoride ion concentration in the composition of from about 0.0025% to about 5.0% or from about 0.005% to about 2.0%, by weight of the oral care composition to provide anticaries effectiveness. A wide variety of fluoride ion-yielding materials can be employed as sources of soluble fluoride in the present invention. Representative fluoride ion sources include: stannous fluoride, sodium fluoride, potassium fluoride, amine fluoride, sodium monofluorophosphate, indium fluoride, amine fluorides such as Olaflur, and many others. Examples of suitable fluoride ion-yielding materials are found in U.S. Pat. No. 3,535,421 to Briner et al. and U.S. Pat. No. 3,678,154 to Widder et al.

Stannous salts include stannous fluoride, stannous chloride, stannous iodide, stannous chlorofluoride, stannous actetate, stannous hexafluorozirconate, stannous sulfate, stannous lactate, stannous tartrate, stannous gluconate, stannous citrate, stannous malate, stannous glycinate, stannous pyrophosphate, stannous metaphosphate, stannous oxalate, stannous phosphate, stannous carbonate, and combinations thereof. Dentifrices containing stannous salts, particularly stannous fluoride and stannous chloride, are described in U.S. Pat. No. 5,004,597 to Majeti et al. Other descriptions of stannous salts are found in U.S. Pat. No. 5,578,293 issued to Prencipe et al. and in U.S. Pat. No. 5,281,410 issued to Lukacovic et al. In addition to the stannous ion source, other ingredients used to stabilize the stannous may be included, such as the ingredients described in Majeti et al. and Prencipe et al.

Zinc salts include zinc fluoride, zinc chloride, zinc iodide, zinc chlorofluoride, zinc actetate, zinc hexafluorozirconate, zinc sulfate, zinc lactate, zinc tartrate, zinc gluconate, zinc citrate, zinc malate, zinc glycinate, zinc pyrophosphate, zinc metaphosphate, zinc oxalate, zinc phosphate, zinc carbonate, and combinations thereof.

Potassium salts include potassium nitrate, potassium citrate, potassium oxalate, potassium bicarbonate, potassium acetate, potassium chloride, and combinations thereof.

In one example, the copper salt can be selected from copper fluoride, copper chloride, copper iodide, copper chlorofluoride, copper actetate, copper hexafluorozirconate, copper sulfate, copper lactate, copper tartrate, copper gluconate, copper citrate, copper malate, copper glycinate, copper pyrophosphate, copper metaphosphate, copper oxalate, copper phosphate, copper carbonate, and combinations thereof. In a further example, the copper salt can be selected from copper gluconate, copper acetate, copper glycinate, and combinations thereof.

Sweeteners can include saccharin, chloro-sucrose (sucralose), steviolglycosides, rebaudioside A, rebaudioside B, rebaudioside C, rebaudioside D, rebaudioside E, rebaudioside F, dulcoside A, dulcoside B, rubusoside, stevia, stevioside, acesulfame K, xylitol, neohesperidine DC, alitame, aspartame, neotame, alitame, thaumatin, cyclamate, glycyrrhizin, mogroside IV, mogroside V, Luo Han Guo sweetener, siamenoside, monatin and its salts (monatin SS, RR, RS, SR), curculin, monellin, mabinlin, brazzein, hemandulcin, phyllodulcin, glycyphyllin, phloridzin, trilobatin, baiyanoside, osladin, polypodoside A, pterocaryoside A, pterocaryoside B, mukurozioside, phlomisoside I, periandrin I, abrusoside A, cyclocarioside I,N-[N-[3-(3-hydroxy-4-methoxyphenyl)propyl]-L-α-aspartyl]-L-phenylalanine 1-methyl ester, N-[N-[3-(3-hydroxy-4-methoxyphenyl)-3-methylbutyl]-L-α-aspartyl]-L-phenylalanine 1-methyl ester, N-[N-[3-(3-methoxy-4-hydroxyphenyl)propyl]-L-α-aspartyl]-L-phenylalanine 1-methyl ester, salts thereof, and combinations thereof.

Rebiana can be a steviolglycoside from Cargill Corp., Minneapolis, Minn., which is an extract from the leaves of the Stevia rebaudiana plant (hereinafter referred to as “Rebiana”). This is a crystalline diterpene glycoside, about 300× sweeter than sucrose. Examples of suitable stevioglycosides which may be combined include rebaudioside A, rebaudioside B, rebaudioside C, rebaudioside D, rebaudioside E, rebaudioside F, dulcoside A, dulcoside B, rubusoside, stevioside, or steviolbioside. According to particularly desirable examples of the present invention, the combination of high-potency sweeteners comprises rebaudioside A in combination with rebaudioside B, rebaudioside C, rebaudioside F, rebaudioside F, stevioside, steviolbioside, dulcoside A. Sweeteners are generally included in an oral care composition at a level of about 0.0005% to about 2%, by total weight of the oral care composition.

Carrier materials can include water, glycerin, sorbitol, polyethylene glycols having a molecular weight of less than about 50,000, propylene glycol and other edible polyhydric alcohols, ethanol, or combinations thereof. The oral care compositions of the present invention include from about 5% to about 80%, by weight of the composition, of a carrier material. In certain examples, the compositions contain carrier materials in an amount of from about 10% to about 40%, by total weight of the oral care composition. In one example, the composition can contain from about 30% to about 95% water, in another example from about 40% to about 90%, in another example from about 50% to about 80%, and in another example from about 60% to about 70%. In another example the composition contains from about 1% to about 20% water, in another example from about 2% to about 15% water, in another example from about 3% to about 10% water, and in another example from about 4% to about 8% water. In one example, the composition contains greater than about 5% water, in another example greater than about 8%, in another example greater than about 10%, in another example greater than about 15%, in another example greater than about 20%, in another example greater than about 25%, in another example greater than about 30%, in another example greater than about 40%, and in another example greater than about 50%.

Antimicrobial agents include quaternary ammonium compounds. Those useful in the present invention include, for example, those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethoylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium antibacterial agents.

Other quaternary ammonium compounds include the pyridinium compounds. Examples of pyridinium quaternary ammonium compounds include bis[4-(R-amino)-1-pyridinium]alkanes as disclosed in U.S. Pat. No. 4,206,215, Jun. 3, 1980, to Bailey and cetylpyridinium and tetradecylpyridinium halide salts (i.e., chloride, bromide, fluoride and iodide).

The oral care compositions of the present invention may also include other antimicrobial agents including non-cationic antimicrobial agents such as halogenated diphenyl ethers, phenolic compounds including phenol and its homologs, mono and poly-alkyl and aromatic halophenols, resorcinol and its derivatives, xylitol, bisphenolic compounds and halogenated salicylanilides, benzoic esters, and halogenated carbanilides. Also useful antimicrobials are enzymes, including endoglycosidase, papain, dextranase, mutanase, and combinations thereof. Such agents are disclosed in U.S. Pat. No. 2,946,725, Jul. 26, 1960, to Norris et al. and in U.S. Pat. No. 4,051,234 to Gieske et al. Examples of other antimicrobial agents include chlorhexidine, and flavor oils such as thymol. In another example, the antimicrobial agent can include triclosan.

The compositions of the present invention may contain antimicrobial agents in an amount of from about 0.035% or more, from about 0.1% to about 2.0%, from about 0.045% to about 1.0%, or from about 0.05% to about 0.10%, by total weight of the oral care composition. In another example from about 0.001% to about 1.5% antimicrobial agent, in another example from about 0.005% to about 0.8%, in another example 0.01% to about 0.7%, in another example from about 0.05% to about 0.5%, and in another example from about 0.1% to about 0.3%.

Non-limiting examples of peroxide compounds can include hydrogen peroxide, urea peroxide, calcium peroxide, sodium peroxide, zinc peroxide, or combinations thereof. In one example, the composition can contain greater than about 0.5% peroxide, in another example greater than about 0.75%, in another example greater than about 1%, in another example greater than about 1.25%, in another example greater than about 1.5%, in another example greater than about 1.75%. in another example greater than about 2%, in another example greater than about 2.25%, in another example greater than about 2.5%, in another example greater than about 2.75%, in another example greater than about 2.85%, in another example greater than about 2.9%, in another example greater than about 2.95%, and in another example greater than about 3%. In another example, the composition can contain from about 0.1% to about 5% peroxide, in another example 0.5% to about 4%, in another example 1% to about 3.5%, in another example about 1.5% to about 3.25%, and in another example about 2% to about 3%. In another example, the composition can contain from about 1% to about 10% peroxide, in another example from about 2% to about 8% peroxide, in another example from about 3% to about 7% peroxide, and in another example from about 4% to about 6% peroxide.

In one example, the oral care composition can include bleaching agents instead of or in addition to peroxides. Bleaching agents can include perborates, percarbonates, peroxyacids, persulfates, and combinations thereof. One example of a percarbonate is sodium percarbonate.

An example of a persulfate includes oxones. Some bleaching agents provide a burn sensation within an oral care composition, for example peroxides and percarbonates.

The compositions of the present invention may contain bleaching agents in an amount of from about 0.01% to about 30%, from about 0.1% to about 10%, or from about 0.5% to about 5%, by total weight of the oral care composition.

Surfactants may include anionic surfactants such as organophosphate, which include alkyl phosphates. These surface active organophosphate agents have a strong affinity for enamel surface and have sufficient surface binding propensity to desorb pellicle proteins and remain affixed to enamel surfaces. Suitable examples of organophosphate compounds include mono-, di- or triesters represented by the general structure below wherein Z1, Z2, or Z3 may be identical or different, at least one being an organic moiety, in one example selected from linear or branched, alkyl or alkenyl group of from 1 to 22 carbon atoms, optionally substituted by one or more phosphate groups; alkoxylated alkyl or alkenyl, (poly)saccharide, polyol or polyether group.

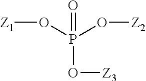

Some other organophosphate agents include alkyl or alkenyl phosphate esters represented by the following structure:

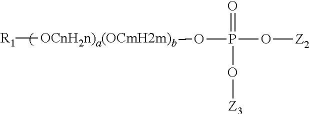

wherein R1 represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z2 and Z3 may be identical or different, each represents hydrogen, alkali metal, ammonium, protonated alkyl amine or protonated functional alkyl amine such as an alkanolamine, or a R1-(OCnH2n)a(OCmH2m)b- group. Examples of suitable agents include alkyl and alkyl (poly)alkoxy phosphates such as lauryl phosphate; PPG5 ceteareth-10 phosphate; Laureth-1 phosphate; Laureth-3 phosphate; Laureth-9 phosphate; Trilaureth-4 phosphate; C12-18 PEG 9 phosphate; Sodium dilaureth-10 phosphate. In one example, the alkyl phosphate is polymeric. Examples of polymeric alkyl phosphates include those containing repeating alkoxy groups as the polymeric portion, in particular 3 or more ethoxy, propoxy, isopropoxy or butoxy groups.

Zwitterionic or amphoteric surfactants useful in the present invention can include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, such as carboxy, sulfonate, sulfate, phosphate or phosphonate. Suitable amphoteric surfactants include betaine surfactants such as disclosed in U.S. Pat. No. 5,180,577 to Polefka et al. Typical alkyl dimethyl betaines include decyl betaine or 2-(N-decyl-N,N-dimethylammonio) acetate, coco betaine or 2-(N-coco-N,N-dimethyl ammonio) acetate, myristyl betaine, palmityl betaine, lauryl betaine, cetyl betaine, stearyl betaine, etc. Amphoteric surfactants useful herein further include amine oxide surfactants. The amidobetaines are exemplified by cocoamidoethyl betaine, cocamidopropyl betaine (CAPB), and lauramidopropyl betaine. The unwanted tastes often associated with these surfactants are soapy, bitter, chemical, or artificial.

Additional suitable polymeric organophosphate agents can include dextran phosphate, polyglucoside phosphate, alkyl polyglucoside phosphate, polyglyceryl phosphate, alkyl polyglyceryl phosphate, polyether phosphates and alkoxylated polyol phosphates. Some specific examples are PEG phosphate, PPG phosphate, alkyl PPG phosphate, PEG/PPG phosphate, alkyl PEG/PPG phosphate, PEG/PPG/PEG phosphate, dipropylene glycol phosphate, PEG glyceryl phosphate, PBG (polybutylene glycol) phosphate, PEG cyclodextrin phosphate, PEG sorbitan phosphate, PEG alkyl sorbitan phosphate, and PEG methyl glucoside phosphate. Suitable non-polymeric phosphates include alkyl mono glyceride phosphate, alkyl sorbitan phosphate, alkyl methyl glucoside phosphate, alkyl sucrose phosphates. The impurities in these phosphates may induce a burning sensation. Impurities may include dodecanol, dodecanal, benzaldehyde, and other TRPA1 or TRPV1 agonists.

Cationic surfactants useful in the present invention can include derivatives of quaternary ammonium compounds having one long alkyl chain containing from about 8 to 18 carbon atoms such as lauryl trimethylammonium chloride, cetyl trimethylammonium bromide, coconut alkyltrimethylammonium nitrite, cetyl pyridinium fluoride, etc. Quaternary ammonium halides having detergent properties can be used, such as those described in U.S. Pat. No. 3,535,421 to Briner et al. Certain cationic surfactants can also act as germicides in the oral care compositions disclosed herein.

Examples of some flavors that may be used in oral care compositions are mint oils, and components thereof, wintergreen, clove bud oil, cassia, sage, parsley oil, marjoram, lemon, orange, propenyl guaethol, heliotropine, cis-4-heptenal, diacetyl, methyl-p-tert-butyl phenyl acetate, methyl salicylate, ethyl salicylate, 1-menthyl acetate, oxanone, α-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, α-terpineol, linalool, limonene, citral, neral, geranial, geraniol nerol, maltol, ethyl maltol, anethole, dihydroanethole, carvone, menthone, β-damascenone, ionone, γ-decalactone, γ-nonalactone, γ-undecalactone, isopulegol, piperitone, or combinations thereof. Generally suitable flavoring ingredients are chemicals with structural features and functional groups that are less prone to redox reactions. These include derivatives of flavor chemicals that are saturated or contain stable aromatic rings or ester groups.

Flavors can be present in an amount of from about 0.4% to about 5%, by total weight of the oral care composition, in another example from about 0.8% to about 4%, in another example from about 1% to about 3.5%, and in another example from about 1.5% to about 3%. In another example, it can be desirable to have a flavor composition at less than about 4%, less than about 3.5%, by total weight of the oral care composition, in another example less than about 3%, and in another example less than about 2%.

Anti-tartar agents can include pyrophosphate salts as a source of pyrophosphate ion. The pyrophosphate salts useful in the present compositions include, for example, the mono-, di- and tetraalkali metal pyrophosphate salts and combinations thereof. Disodium dihydrogen pyrophosphate (Na2H2P2O7), sodium acid pyrophosphate, tetrasodium pyrophosphate (Na4P2O7), and tetrapotassium pyrophosphate (K4P2O7) in their unhydrated as well as hydrated forms are further species. In compositions of the present invention, the pyrophosphate salt may be present in one of three ways: predominately dissolved, predominately undissolved, or a combination of dissolved and undissolved pyrophosphate. The amount of pyrophosphate salt useful in making these compositions is any tartar control effective amount. In varying examples, the amount of pyrophosphate salt may be from about 1.5% to about 15%, from about 2% to about 10%, or about 3% to about 8%, by total weight of the oral care composition.

Examples of some colorants that may be used in oral care compositions include D&C Yellow No. 10, FD&C Blue No. 1, FD&C Red No. 40, D&C Red No. 33 and combinations thereof. In certain examples, the composition comprises colorant in an amount of from about 0.0001% to about 0.1% or from about 0.001% to about 0.01%, by weight of the oral care composition. Some colorants provide an unwanted taste, for example, D&C Red No. 33. The unwanted tastes often associated with this colorant are metallic, sharp, or chemical. Colorants are generally present in an amount of from about 0.001% to about 0.5%, by weight of the oral care composition.

Sensates may also be part of an oral care composition. Sensate molecules such as cooling, warming, and tingling agents are useful to deliver signals to the user. Sensates are generally present in an amount of from about 0.001% to about 2%, by weight of the oral care composition. The most well-known cooling sensate compound can be menthol, particularly L-menthol, which is found naturally in peppermint and spearmint oils notably of Mentha piperita, Mentha arvensis L and Mentha viridis L. Other isomers of menthol (neomenthol, isomenthol and neoisomenthol) have somewhat similar, but not identical odor and taste, for instance having disagreeable odor and taste described as earthy, camphor, musty, etc. The biggest difference among the isomers is in their cooling potency. L-menthol provides the most potent cooling, by having the lowest cooling threshold of about 800 ppb, which is the concentration level where the cooling effect can be clearly recognized. At this level, there can be no cooling effect for the other isomers. For example, d-neomenthol is reported to have a cooling threshold of about 25,000 ppb and 1-neomenthol about 3,000 ppb.

Of the menthol isomers the 1-isomer occurs most widely in nature and is typically what is referred by the name menthol having coolant properties. L-menthol has the characteristic peppermint odor, has a clean fresh taste and exerts a cooling sensation when applied to the skin and mucosal surfaces.

Among synthetic coolants, many are derivatives of or are structurally related to menthol, for example containing the cyclohexane moiety, and derivatized with functional groups including carboxamide, ketal, ester, ether and alcohol. Examples include the ρ-menthanecarboxamide compounds such as N-ethyl-ρ-menthan-3-carboxamide, known commercially as “WS-3”, and others in the series such as WS-5 (N-ethoxycarbonylmethyl-ρ-menthan-3-carboxamide), WS-12 (1R*,2S*)—N-(4-Methoxyphenyl)-5-methyl-2-(1-methylethyl)cyclohexanecarboxamidel and WS-14 (N-tert-butyl-ρ-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”. Additional examples of synthetic coolants include alcohol derivatives such as 3-(1-menthoxy)-propane-1,2-diol known as TK-10, isopulegol (under the tradename Coolact P) and p-menthane-3,8-diol (under the tradename Coolact 38D) all available from Takasago Corp., Tokyo, Japan; menthone glycerol acetal known as MGA; menthyl esters such as menthyl acetate, menthyl acetoacetate, menthyl lactate known as Frescolat® supplied by Symrise AG, Holzminden, Germany, and monomenthyl succinate under the tradename Physcool from V. Mane FILS, Notre Dame, France. TK-10 is described in U.S. Pat. No. 4,459,425 to Amano et al. Other alcohol and ether derivatives of menthol are described in GB 1,315,626 and in U.S. Pat. Nos. 4,029,759; 5,608,119; and 6,956,139. WS-3 and other carboxamide cooling agents are described in U.S. Pat. Nos. 4,136,163; 4,150,052; 4,153,679; 4,157,384; 4,178,459 and 4,230,688.

Additional N-substituted p-menthane carboxamides are described in WO 2005/049553A1 including N-(4-cyanomethylphenyl)-p-menthanecarboxamide, N-(4-sulfamoylphenyl)-p-menthanecarboxamide, N-(4-cyanophenyl)p-menthanecarboxamide, N-(4-acetylphenyl)-p-menthanecarboxamide, N-(4-hydroxymethylphenyl)-p-menthanecarboxamide and N-(3-hydroxy-4-methoxyphenyl)-p-menthanecarboxamide. Other N-substituted p-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N-((5-methyl-2-(1-methylethyl)cyclohexyl)carbonyl)alanine ethyl ester. Menthyl esters including those of amino acids such as glycine and alanine are disclosed e.g., in EP 310,299 and in U.S. Pat. Nos. 3,917,613; 3,991,178; 5,703,123; 5,725,865; 5,843,466; 6,365,215; and 6,884,903. Ketal derivatives are described, e.g., in U.S. Pat. Nos. 5,266,592; 5,977,166; and 5,451,404. Additional agents that are structurally unrelated to menthol but have been reported to have a similar physiological cooling effect include alpha-keto enamine derivatives described in U.S. Pat. No. 6,592,884 including 3 -methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-12-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H. R. Watson, et al. J. Soc. Cosmet. Chem. (1978), 29, 185-200 and R. Eccles, J. Pharm. Pharmacol., (1994), 46, 618-630 and phosphine oxides as reported in U.S. Pat. No. 4,070,496.

Some examples of warming sensates include ethanol; capsicum; nicotinate esters, such as benzyl nicotinate; polyhydric alcohols; capsicum powder; a capsicum tincture; capsicum extract; capsaicin; homocapsaicin; homodihydrocapsaicin; nonanoyl vanillyl amide; nonanoic acid vanillyl ether; vanillyl alcohol alkyl ether derivatives such as vanillyl ethyl ether, vanillyl butyl ether, vanillyl pentyl ether, and vanillyl hexyl ether; isovanillyl alcohol alkyl ethers; ethylvanillyl alcohol alkyl ethers; veratryl alcohol derivatives; substituted benzyl alcohol derivatives; substituted benzyl alcohol alkyl ethers; vanillin propylene glycol acetal; ethylvanillin propylene glycol acetal; ginger extract; ginger oil; gingerol; zingerone; or combinations thereof. Warming sensates are generally included in an oral care composition at a level of about 0.05% to about 2%, by weight of the oral care composition.

Abrasive polishing material can be any material that does not excessively abrade dentin. The oral care compositions of the present invention may comprise abrasive polishing material in an amount of from about 6% to about 70% or from about 10% to about 50%, by weight of the oral care composition. Typical abrasive polishing materials can include silicas including gels and precipitates; aluminas; phosphates including orthophosphates, polymetaphosphates, and pyrophosphates; and mixtures thereof. Specific examples include silicone microspheres such as polyorganosilsesquioxane particles, dicalcium orthophosphate dihydrate, calcium pyrophosphate, tricalcium phosphate, calcium polymetaphosphate, insoluble sodium polymetaphosphate, rice hull silica, hydrated alumina, beta calcium pyrophosphate, calcium carbonate, and resinous abrasive materials such as particulate condensation products of urea and formaldehyde, and others such as disclosed by Cooley et al in U.S. Pat. No. 3,070,510. In certain examples, if the oral composition or particular phase comprises a polyphosphate having an average chain length of about 4 or more, calcium containing abrasives and alumina are not preferred abrasives. In certain examples, the composition is substantially free of silica.

In another example, the composition can contain a silica abrasive. Silica abrasive polishing materials that may be used in the present invention, as well as other abrasives, generally have an average particle size ranging between about 0.1 to about 30 μm or from about 5 to about 15 μm. The abrasive can be precipitated silica or silica gels such as the silica xerogels described in Pader et al., U.S. Pat. No. 3,538,230 and DiGiulio, U.S. Pat. No. 3,862,307. Silica xerogels marketed under the trade name “Syloid” by the W.R. Grace & Company, Davison Chemical Division, Augusta, Ga. may be used. Also precipitated silica materials such as those marketed by the J. M. Huber Corporation, Edison, N.J. under the trade name, “Zeodent”, particularly the silica carrying the designation “Zeodent 119”, may be used. The types of silica dental abrasives useful in the oral care compositions of the present invention are described in more detail in U.S. Pat. Nos. 4,340,583; 5,589,160; 5,603,920; 5,651,958; 5,658,553; and 5,716,601.

In one example, the abrasive can include polyorganosilsesquioxane particles. The types of polyorganosilsesquioxane particles useful in the oral care compositions of the present invention are described in more detail in U.S. patent application Ser. No. 14/249,650.

In another example, the abrasive can include calcium pyrophosphate. In another example, the abrasive can include poly(methyl methacrylate), calcium carbonate, dicalcium phosphate, and/or barium sulfate.

Thickening material or binders may be used to provide a desirable consistency to the oral care compositions of the present invention.

Thickening materials can include carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthan gum, gum arabic, and gum tragacanth can also be used. Colloidal magnesium aluminum silicate or finely divided silica can be used as part of the thickening material to further improve texture. Thickening materials can be used in an amount from about 0.1% to about 15%, by weight of the oral care composition. The oral care compositions can also contain binders that can also adjust formulation texture and mouth feel.

In another example, the thickening agent can include the addition of polymers of acrylic acid crosslinked with an unsaturated polyfunctional agent such as a polyallyl ether of sucrose. These carboxy vinyl polymers have the CTFA (Cosmetic, Toiletry and Fragrance Association) adopted name of “carbomer.” A carbomer can include negatively charged polyelectrolytes, such as Carbomer 956 (available from Lubrizol Corporation, Wickliffe, Ohio). In another example the carbomer can be selected from the group consisting of acrylates/C10-30 alkyl acrylate crosspolymer, sodium polyacrylate; polyacrylate-1 Crosspolymer (available from Lubrizol); polyacrylate Crosspolymer-11 (available from Clariant, Inc., Louisville, Ky., USA), acrylates/C10-30 alkyl acrylate crosspolymer, and combinations thereof. In one example, the carbomer can be Carbomer 956. In one example, the composition contains from about 0.1% to about 15% carbomer, in another example from about 0.3% to about 10% carbomer, in another example from about 0.5% to about 6% carbomer, in another example from about 0.7% to about 3% carbomer, and in another example from about 0.9% to about 1.5% carbomer. Examples of additional carbomers can be found in U.S. Pat. No. 2,798,053.

Humectants keep oral care compositions from hardening upon exposure to air and certain humectants can also impart desirable sweetness of flavor to dentifrice compositions. Suitable humectants for use in the present invention include glycerin, sorbitol, polyethylene glycol, propylene glycol, xylitol, and other edible polyhydric alcohols. The oral care compositions of the present invention may comprise humectants in an amount of from about 0% to about 70% or from about 15% to about 55%, by weight of the oral care composition.

### Brookfield Viscosity Test

The viscometer is Brookfield viscometer, Model ½ RVT, with a Brookfield “Heliopath” stand (available from Brookfield Engineering Laboratories, Middleboro, Mass.). The spindle is a conventional “E-series” T-shaped spindle. The viscometer is placed on the Heliopath stand and leveled via spirit levels. The E spindle is attached, and the viscometer is set to 2.5 RPM while it is running The viscosity is measured after 1 minute and the temperature is constant at 25° C. The “Brookfield Unit” in which results obtained from this method have traditionally been expressed is simply the direct readout of the instrument under standard conditions, i.e., using the “E” spindle at 2.5 RPM, or calculated equivalent.

Total Flavor Display Test by Headspace GC-MS

Samples for flavor display analysis are prepared by placing one gram of test dentifrice into a 20-mL headspace vial (Wheaton® vial part number 16-2000 with Wheaton® cap, part number 16-0050m, Wheaton® Industries Incorporated, Millville, N.J., USA). Additionally, 3 mL of artificial saliva is added to each 20-mL vial containing each dentifrice sample. The artificial saliva solution is comprised of 20 mM NaHCO₃, 2.75 mM K₂HPO₄, 12.2 mM KH₂PO₄, and 15 mM NaCl at pH of 7.0 dissolved in distilled water. A stir bar is added to the vial before capping the vial and placing it onto a Gerstel™ MultiPurpose Sampler MPS2 tray (VT32-20, Gerstel™ Incorporated, USA, Linthicum, Md., USA). Prior to GC-MS analysis, each sample, in sequence, is incubated for 1 minute at 37° C. with stirring at 400 rpm in the agitator/stirrer component of the Gerstel™ sampler. These conditions were selected to approximate flavor release during consumer brushing and also to create good dispersion and foaming of the dentifrice.

At the one minute time point, one mL of the headspace is sampled from above the foam level in the closed vial by piercing the septum with a headspace syringe. The sampled headspace is then injected into an Agilent™ 7890 gas chromatograph (GC) equipped with an HP-FFAP column (30 m×0.25 mm ID×0.25 μm film thickness; Agilent part number 19091F-433) that is connected to an Agilent™ 5975C mass spectrometry detector (MSD), all from Agilent™ Technologies, Wilmington, Del., USA.

Chromatographic separation of the flavors contained within the headspace sample is achieved with the following conditions: inlet temperature 250° C.; split ratio 10:1; column flow 1.4 mL helium/minute; oven temperature program 40° C. for 0.5 minutes, then 15° C./minute to 240° C. and hold at 240° C. for 1.5 minutes. As flavor components elute from the column, they are detected with mass spectrometric conditions as follows: electron ionization at 70 electron volts; transfer line temperature held at 250° C.; source temperature held at 230° C.; quadrupole temperature held at 150° C.; and mass spectrometer operated in scan mode from mass-to-charge ratio 35 to 350 amu. Each flavor compound is identified via retention time and mass spectral fragmentation pattern and peak areas are recorded for each.

Peak areas for all flavor components are then summed to obtain a total flavor peak area. For each dentifrice sample, the entire sample preparation and analysis procedure is performed in triplicate, with all dentifrice samples and their replicates randomized and run within a single continuous batch. The average total flavor peak area obtained from each dentifrice sample is compared among the dentifrice samples analyzed within the same analysis batch. Peak areas should only be compared for dentifrice products analyzed within a given batch to ensure accurate conclusions about relative flavor display. Peak areas generated during different analysis periods or on different instrumentation should not be compared, due to the potential for longer term drift or differences in absolute GC-MS instrumental response.

### Peroxide Stability Test

The following method is used to determine the stability of the peroxide in an oral care composition. First, 0.4000 g (+/−0.02 g) of the oral care sample is added to a 250 ml plastic beaker. This procedure is performed in duplicate. Then, 100 mL of 0.04N H₂50₄ (sulfuric acid) is added to the beaker. A stir bar is added, the beaker covered with a plastic paraffin film such as Parafilm M® (available from Bemis® Flexible Packaging, Oshkosh, Wis.), and the solution is stirred for at least ten minutes (and possibly much longer) until the sample looks homogeneous. After stirring, 25 mL of 10% KI (potassium iodide) solution and 3 drops of NH4-Molybdate are added to the beaker, the beaker is covered with the plastic paraffin film, and the solution is stirred for another 3-20 minutes. The resulting solution is analyzed via autotitration with 0.1N sodium-thiosulfate solution. The % hydrogen peroxide is calculated using the following formula:

$$\% Hydrogen Peroxide = \frac{\begin{matrix}Thiosulfate used ( mL ) \times \\ Thiosulfate Normality ( \frac{meq}{mL} ) \times 1.701 ( g / meq )\end{matrix} }{Sample weight ( g )}$$

Measurements were taken after the initial making of the formulation and then again after the formulation was stored in a non-reactive vessel for a given time period at 40° C. Peroxide stability was calculated as the peroxide percent measured after a given time period at 40° C. divided by the initial peroxide percent measured, then multiplied by 100. Product placed at 40° C. represents accelerated stability and can be used to determine how the oral care compositions may react throughout their shelf life. The given time can be any suitable amount of time. For instance four weeks (28 days) or eight weeks (56 days) or 13 weeks or 26 weeks.

### pH Test Method

First, calibrate the Thermo Scientific Orion 320 pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 7 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 4 buffer and wait until the pH icon stops flashing and press the measure button. Rinse the electrode with distilled water and carefully wipe with a scientific cleaning wipe. Now the pH meter is calibrated and can be used to test the pH of a solution.

The pH of the liquid medication is measured using the calibrated pH meter at ambient temperature.

| | Ex. 1 | Ex. 2 | Ex. 3 |
|---|---|---|---|
| | (wt. %) | (wt. %) | (wt. %) |
| Lanette ® W¹ | 0 | 1.000 | 13.000 |
| SepiMAX ™ ZEN² | 3.000 | 2.000 | 0 |
| Sodium Lauryl Sulfate Powder | 0.100 | 0.100 | 2.600 |
| Water | 67.887 | 67.890 | 56.435 |
| Sodium Fluoride | 0.243 | 0.243 | 0.243 |
| Sodium acid pyrophosphate | 0.300 | 0.300 | 0.300 |
| Disodium phosphate | 0.200 | 0.200 | 0.200 |
| Sucralose | 0.400 | 0.400 | 0.500 |
| Sodium Lauryl Sulfate Powder | 1.200 | 1.200 | 0 |
| Tospearl ® 145³ | 15.000 | 15.000 | 15.000 |
| H₂O₂ (35% solution)⁴ | 8.570 | 8.570 | 8.570 |
| Flavor | 3.000 | 3.000 | 3.000 |
| Phosphoric Acid (approximate) | 0.100 | 0.100 | 0.150 |

¹Lanette ® W is mixture (40:40:10) of cetyl alcohol/stearyl alcohol/sodium lauryl sulfate and is available from BASF Corp.
²Available from SEPPIC
³Polyorganosilsequioxane particles, more specifically polymethylsilsesquioxane silicone resin particles, available from Momentive ™ Performance Materials, New York, USA.
⁴8.57% of a 35% hydrogen peroxide (H₂O₂) solution is equivalent to about 3% active H₂O₂

| | Ex. 4 | Ex. 5 | Ex. 6 | Ex. 7 | Ex. 8 |
|---|---|---|---|---|---|
| | (wt. %) | (wt. %) | (wt. %) | (wt. %) | (wt. %) |
| Lanette ® W | 0 | 1.6 | 3.2 | 0 | 1.6 |
| SepiMAX ™ ZEN | 3.2 | 1.6 | 0 | 1.6 | 0 |
| Sodium Lauryl | 0.1 | 0.1 | 0.1 | 0.1 | 0.1 |
| Sulfate Powder | | | | | |
| Water | 69.157 | 69.177 | 69.177 | 70.747 | 70.767 |
| Sodium Fluoride | 0.243 | 0.243 | 0.243 | 0.243 | 0.243 |
| Sodium acid | 0.3 | 0.3 | 0.3 | 0.3 | 0.3 |
| pyrophosphate | | | | | |
| Disodium | 0.2 | 0.2 | 0.2 | 0.2 | 0.2 |
| phosphate | | | | | |
| Sucralose | 0.4 | 0.4 | 0.4 | 0.4 | 0.4 |
| Sodium Lauryl | 1.14 | 1.14 | 1.14 | 1.14 | 1.14 |
| Sulfate Powder | | | | | |
| Tospearl ® 145 | 15 | 15 | 15 | 15 | 15 |
| H₂O₂ (35% | 8.57 | 8.57 | 8.57 | 8.57 | 8.57 |
| solution) | | | | | |
| Flavor | 1.5 | 1.5 | 1.5 | 1.5 | 1.5 |
| Phosphoric acid | 0.19 | 0.17 | 0.17 | 0.2 | 0.18 |
| (pH trim) | | | | | |
| target pH | 4.5-5.0 | 4.5-5.0 | 4.5-5.0 | 4.5-5.0 | 4.5-5.0 |

| | Ex. 9 | Ex. 10 |
|---|---|---|
| | (wt. %) | (wt. %) |
| Water | 68.17 | 68.17 |
| SepiMAX ™ ZEN | 2.50 | 1.25 |
| Carbomer 956⁵ | 0 | 1.25 |
| Sodium Lauryl Sulfate | 0.10 | 0.10 |
| Powder | | |
| MFP | 0.76 | 0.76 |
| Sodium acid | 1.00 | 1.00 |
| pyrophosphate | | |
| Disodium phosphate | 0.20 | 0.20 |
| Sodium Lauryl Sulfate | 1.40 | 1.40 |
| Powder | | |
| Sucralose | 0.30 | 0.30 |
| Calcium | 15.00 | 15.00 |
| Pyrophosphate | | |
| H₂O₂ (35% solution)⁶ | 8.57 | 8.57 |
| Flavor | 2.00 | 2.00 |
| NaOH (50%) (pH | 0 | 0.8 |
| trim) | | |
| Phosphoric Acid (pH | 0 | 0.4 |
| trim) | | |
| target pH >/=5.1 but <5.5 | 5.1-5.5 | 5.1-5.5 |

⁵Available from Lubrizol Corporation, Wickliffe, Ohio
⁶8.57% of a 35% hydrogen peroxide (H₂O₂) solution is equivalent to about 3% active H₂O₂

Examples 1, 4, 7, and 9 were made as follows. First, a first portion of sodium lauryl sulfate (SLS) powder was added to water and mixed until incorporated in a beaker with an overhead mixer. Then, SepiMAX™ ZEN was gently added to water/SLS mixture and as the water thickens, the mixing speed was increased. The SepiMAX™ ZEN/water/SLS solution was mixed for about 20-25 minutes or until the SepiMAX™ ZEN and SLS were both hydrated and a clear solution was formed. Then, the remaining ingredients, including the remaining SLS powder, if present, were added and the remaining ingredients were added individually and mixed. Most, if not all, of the phosphoric acid was added prior to adding the hydrogen peroxide.

Examples 2, 3, and 5 were made according to the procedure for Examples 1, 4, 7, and 9 above. After the SepiMAX™ ZEN and SLS were hydrated and in solution and the Lanette® W was added and mixed. While mixing, the solution was heated to about 70° C., which was above the melting point for Lanette® W. The mixing was continued until the solution was cooled to room temperature. Then, the remaining ingredients were added individually and mixed. Most, if not all, of the phosphoric acid may be added prior to adding the hydrogen peroxide.

Examples 6 and 8 were made according to the procedure for Examples 2 and 5, however only the SLS was hydrated, as no SepiMAX™ ZEN was added to these formulations.

Example 10 was made according to the procedure for Examples 2, 3, and 5, and the Carbomer was added at the same time as the SepiMAX ZEN™.

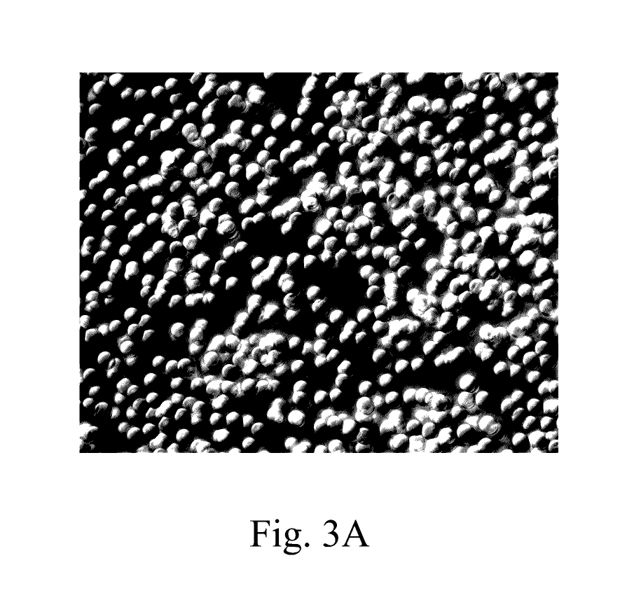

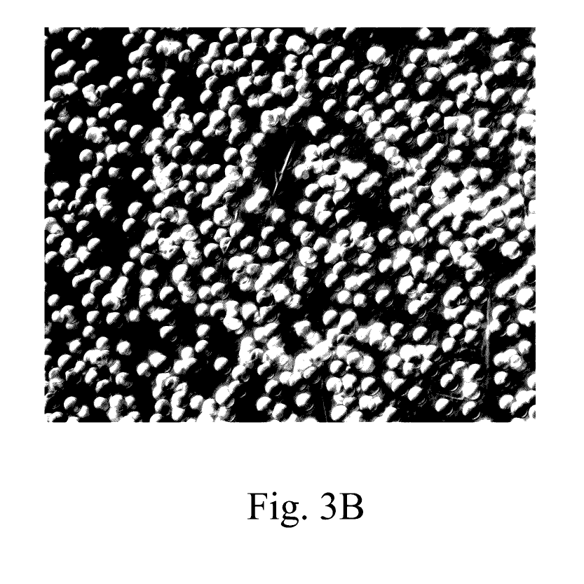

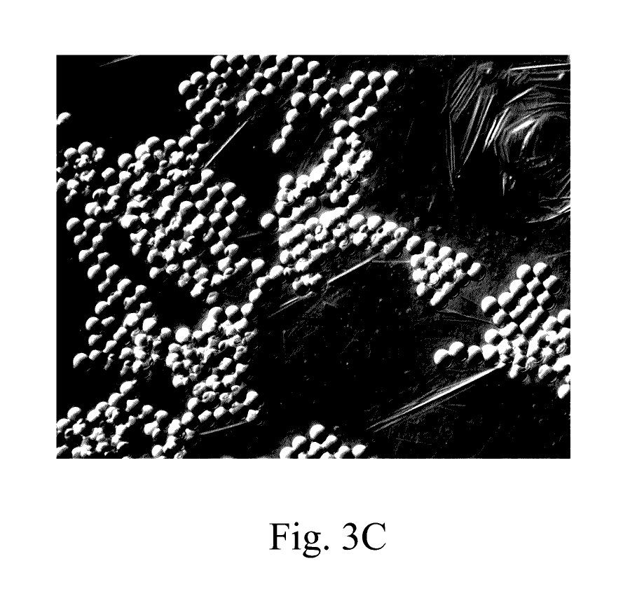

FIG. 3A is a photo at 600× magnification of a composition containing Tospearl® Silicone Microspheres suspended in a formulation thickened with polyacrylate crosspolymer-6, similar to Example 4. FIG. 3A shows a good dispersion and product stability, which is visible because the silicone microspheres are well dispersed and not clumped together. FIG. 3B is a photo at 600X magnification of a composition containing Tospearl® Silicone Microspheres suspended in a formulation containing polyacrylate crosspolymer-6 and a fatty alcohol, similar to Example 5. FIG. 3B shows a good dispersion and viscosity stability, which is visible because the silicone microspheres are not clumped together. FIG. 3C is a photo of a composition at 600× magnification of a composition containing Tospearl® Silicone Microspheres suspended in a formulation containing a fatty alcohol and a surfactant system, similar to Example 6. FIG. 3C shows Tospearl® particles that are clumped together and are not evenly distributed, this indicates that there may be a lack of viscosity stability with this product. In FIG. 3C, the silicone microspheres are clumped together and the fatty alcohol and/or fatty amphilphile have formed vesicles, which may make it difficult for the silicone microspheres as well as other components to disperse.

Values disclosed herein as ends of ranges are not to be understood as being strictly limited to the exact numerical values recited. Instead, unless otherwise specified, each numerical range is intended to mean both the recited values and any real numbers including integers within the range. For example, a range disclosed as “1 to 10” is intended to mean “1, 2, 3, 4, 5, 6, 7, 8, 9, and 10” and a range disclosed as “1 to 2” is intended to mean “1.1, 1.2, 1.3, 1.4, 1.5, 1.6, 1.7, 1.8, 1.9, and 2.”

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
a) a fluoride ion source selected from the group consisting of stannous fluoride, sodium fluoride, potassium fluoride, amine fluoride, sodium monofluorophosphate, indium fluoride, amine fluorides, and combinations thereof;
b) greater than about 0.5% peroxide selected from the group consisting of hydrogen peroxide, urea peroxide, calcium peroxide, sodium peroxide, zinc peroxide, or combinations thereof; and
c) a 2-acrylamido-2-methylpropane sulfonic acid polymer, copolymer, and/or cross polymer.

2. The composition of claim 1 wherein the 2-acrylamido-2-methylpropane sulfonic acid polymers, copolymers, and/or cross polymers is polyacrylate crosspolymer-6.

3. The composition of claim 2 wherein the composition comprises from about 0.1% to 10% polyacrylate crosspolymer-6.

4. The composition of claim 1 wherein the peroxide is hydrogen peroxide.

5. The composition of claim 10 wherein at least about 70% of peroxide remains after 4 weeks according to the Peroxide stability test.

6. The composition of claim 1 further comprising from about 0.5% to about 7% fatty alcohol.

7. The composition of claim 1 further comprising from about 0.5% to about 6% carbomer.

8. The composition of claim 1 comprising from about 1% to about 3.5% flavor.

9. The composition of claim 8 wherein the oral care composition has an average total flavor peak area of greater than about 10% greater than the average total flavor peak area from the gel network chassis containing the exact same flavor at the same level, as determined by the Total Flavor Display Test by Headspace GC-MS.

10. An oral care composition comprising:
a) greater than about 0.5% peroxide selected from the group consisting of hydrogen peroxide, urea peroxide, calcium peroxide, sodium peroxide, zinc peroxide, or combinations thereof;
b) a 2-acrylamido-2-methylpropane sulfonic acid polymers, copolymers, or cross polymers;
c) a flavor;
wherein the oral care composition has an average total flavor peak area of greater than about 10% greater than the average total flavor peak area from the gel network chassis according to the Total Flavor Display Test by Headspace GC-MS.

11. The composition of claim 10 wherein the oral care composition has an average total flavor peak area of 15% greater than the average total flavor peak area from the gel network chassis according to the Total Flavor Display Test by Headspace GC-MS.

12. The composition of claim 10 wherein at least about 70% of peroxide remains after 4 weeks according to the Peroxide stability test.

13. The composition of claim 10 wherein the oral care composition is a dentifrice and the composition further comprises an abrasive selected from the group consisting of silica, polyorganosilsequioxane, calcium pyrophosphate, poly(methyl methacrylate), calcium carbonate, dicalcium phosphate, barium sulfate, and combinations thereof.

14. The composition of claim 10 wherein the composition has a pH from about 4 to about 6.

15. The composition of claim 10 wherein the viscosity is from about 10 BKUs to about 70 BKUs.

16. An oral care composition comprising:
a) a fluoride ion source selected from the group consisting of stannous fluoride, sodium fluoride, potassium fluoride, amine fluoride, sodium monofluorophosphate, indium fluoride, amine fluorides, and combinations thereof;
b) greater than about 0.5% peroxide selected from the group consisting of hydrogen peroxide, urea peroxide, calcium peroxide, sodium peroxide, zinc peroxide, or combinations thereof;
c) an abrasive selected from the group consisting of silica, polyorganosilsequioxane, calcium pyrophosphate, poly(methyl methacrylate), calcium carbonate, dicalcium phosphate, barium sulfate, and combinations thereof;
d) greater than about 10% water;
e) a flavor;
f) a sweetener;
wherein the oral care composition has an average total flavor peak area of greater than about 10% greater than the average total flavor peak area from the gel network chassis containing the exact same flavor at the same level as determined by the Total Flavor Display Test by Headspace GC-MS;
wherein at least about 70% of peroxide remains after 4 weeks according to the Peroxide stability test; and
wherein the oral care composition is a dentifrice.

17. The oral care composition of claim 16 wherein the oral care composition has an average total flavor peak area of greater than about 20% greater than the average total flavor peak area from the gel network chassis containing the exact same flavor at the same level as determined by the Total Flavor Display Test by Headspace GC-MS.

18. The composition of claim 16 herein the fluoride ion source is stannous fluoride.

19. The composition of claim 16 wherein the fluoride ion source is sodium fluoride.

20. The composition of claim 16 comprising greater than 1% of the peroxide source and wherein the peroxide source comprises hydrogen peroxide.
